(19) United States
(12) Patent Application Publication
(10) Pub. No.: US 2025/0213194 A1
(43) Pub. Date: Jul. 3, 2025

(54) MEDICAL CAPSULE SUBSYSTEM FOR PRECISE AUTONOMOUS LOCALIZATION USING ODOMETRY

(71) Applicant: Brigham Young University, Provo, UT (US)

(72) Inventors: Benjamin Terry, Provo, UT (US); Luke Taylor, Provo, UT (US)

(21) Appl. No.: 19/005,854

(22) Filed: Dec. 30, 2024

Related U.S. Application Data
(60) Provisional application No. 63/615,514, filed on Dec. 28, 2023.

(51) Int. Cl. A61B 5/00; A61B 5/06; A61B 5/07

(52) U.S. Cl. CPC A61B 5/6861; A61B 5/065; A61B 5/073; A61B 2562/12

(57) ABSTRACT

Systems and methods of providing an ingestible medical capsule (a capsule) are described. In some implementations, the capsule is configured to measure a distance traveled (by the capsule) through a gastrointestinal tract of a subject. In some cases, the capsule includes an anchor configured to associate with a portion of a gastrointestinal tract of the subject. In some iterations, the capsule includes a capsule body coupled to the anchor via a strand. In some cases, the capsule body is configured to travel a distance away from the anchor along the gastrointestinal tract of the subject and to measure the distance using the strand as a reference. Additional implementations are described.

### RELATED APPLICATION

This application claims priority to U.S. Provisional Patent Application No. 63/615,514, entitled A MEDICAL CAPSULE SUBSYSTEM FOR PRECISE AUTONOMOUS LOCALIZATION USING ODOMETRY, which was filed on Dec. 28, 2023; the entire disclosure of which is incorporated herein by reference.

### FIELD

The present disclosure relates to medical devices, and more particularly to diagnostic medical devices, including ingestible diagnostic and therapeutic devices.

### BACKGROUND

Some traditional systems and methods for diagnosing and treating ailments of the gastrointestinal (GI) tract have one or more drawbacks. In this regard, they can be uncomfortable, expensive, time-consuming, invasive, difficult to use, inaccurate, or otherwise undesirable. For example, endoscopy is a medical procedure often used to examine the interior of organs and cavities in the body using an endoscope (a flexible tube with various instruments, such as a light and camera, attached). It is often used to diagnose conditions, such as gastrointestinal issues, and sometimes to perform procedures like removing polyps or taking tissue biopsies. However, drawbacks include potential complications like bleeding, infection, or perforation of organs. Additionally, the procedure can be uncomfortable for patients, and sometimes there is a risk of incomplete visualization of the area being examined due to the endoscope's physical constraints.

Thus, while techniques currently exist that are used to diagnose and treat GI tract conditions, challenges still exist, including those listed above. Accordingly, it would be an improvement in the art to augment or even replace current techniques with other techniques.

### SUMMARY

According to some implementations of the presently described systems and methods, one or more ingestible medical capsules (capsules) are provided for use in non-invasive diagnosis and treatment of disease (such as GI tract diseases), research, or other uses. The ingestible medical capsule can include any component configured to assist in the diagnosis or treatment of disease, but some implementations are configured to deliver pharmaceuticals or other compounds; collect and transmit images or other data; implant one or more sensors or other devices; collect one or more samples (e.g., tissue samples, gut microbiome samples, fecal samples, or other samples); target one or more specific areas of a patient; measure one or more portions of a GI tract; or otherwise assist in diagnostic, treatment, or research functions.

In some implementations, accurate and precise localization techniques greatly enhance the functionality of the capsule. Although it may be possible to determine an approximate location of some previously existing ingestible devices (e.g., generally in the area of the stomach, versus generally in the area of the colon), some implementations of the capsule according to the present disclosure provide advantages over some methods of general localization, at least in that they are configured to measure (and transmit) much more precise localization data. In some implementations, in addition to measuring more precise localization data, the capsule can be configured to take one or more additional actions (e.g., treatment actions) based on the more-precise localization data.

In some implementations, the capsule is configured to measure a linear distance travelled by the capsule (e.g., using odometry or any other suitable method). Although some implementations can utilize algorithms as part of the localization process, some implementations of the capsule utilize one or more mechanical approaches to distance tracking, allowing them to avoid complex algorithms needed by some other localization methods. Further, some implementations of the capsule are able to determine localization independently of any input from extemal systems (although some iterations are able to utilize external input as well). In some cases, the systems and methods have less of a negative impact on the subject, as large sensor arrays around the subject's abdomen are not necessary (although, once again, they may be used in connection with some embodiments). Moreover, some instances allow for a simplified data transmission process, as (in contrast to some localization techniques) a time and a direction of a signal (e.g., a radiofrequency (RF) signal or any other type of externally detectable signal) do not need to be precisely measured, since the signal itself can carry the localization data (as opposed to trying to use the signal to calculate the localization).

By way of example, according to some iterations of the capsule, the capsule includes a spool wound with a strand (e.g., a spool wound with a dissolvable suture thread or any other suitable material). In some cases, the spool is configured to rotate as the capsule traverses a portion of the GI tract, thereby leaving behind a trail of the strand. In various implementations, the rotations of the spool are tracked by the capsule (or any other suitable component) and mapped to a linear distance traveled by the capsule. Some implementations of the capsule utilizing such a tracking method are able to accurately track distances of up to 10.5 m or more, with very low error margins.

The skilled artisan will recognize that many variations are possible, and that additional features may be useful in accomplishing the objectives of the systems and methods provided herein. Other iterations of the systems and methods are also described below.

### DESCRIPTION OF THE FIGURES

The objects and features of the present disclosure will become more fully apparent from the following description and appended claims, taken in conjunction with the accompanying figures. Understanding that these figures depict only some embodiments of the disclosed systems and methods and are, therefore, not to be considered limiting in scope, the systems and methods will be described and explained with additional specificity and detail through the use of the accompanying figures in which:

FIG. 1 shows a cross-sectional front perspective view of an ingestible medical capsule, in accordance with a representative embodiment of the disclosed systems and methods;

FIG. 2 shows a perspective, exploded, cross-sectional view of a portion of the ingestible medical capsule, in accordance with a representative embodiment;

FIG. 3A-3D show an intestinal track to illustrate some functionality of the ingestible medical capsule, in accordance with a representative embodiment;

FIG. 4 shows a transmission testing setup for the ingestible medical capsule, in accordance with a representative embodiment;

FIG. 5 shows a graph relating to a distance model adjustment process, in accordance with a representative embodiment;

FIGS. 6A-6B each provide a diagram showing the ingestible medical capsule anchored to a cheek of a subject in accordance with some representative embodiments; and

FIGS. 6C-6D each provide a diagram showing the ingestible medical capsule disposed within a gastrointestinal tract of the subject, where the ingestible medical capsule is anchored to the subject prior to and outside of a stomach of the patient (e.g., anchored to a mouth or face of the subject), in accordance with some embodiments.

### DETAILED DESCRIPTION

According to some embodiments of the present systems and methods, one or more ingestible medical capsules 10 are provided for use in non-invasive diagnosis and treatment of disease (such as GI diseases), research, or other purposes. Examples of diseases the capsule can target include Crohn's disease, irritable bowel syndrome (IBS), ulcerative colitis, gastroesophageal reflux disease (GERD), esophagitis, Barrett's esophagus, esophageal cancer, achalasia, esophageal strictures, gastritis, peptic ulcers, stomach cancer, gastroparesis, celiac disease, small intestinal bacterial overgrowth (SIBO), intestinal obstruction, inflammatory bowel disease (IBD), diverticulosis, diverticulitis, colorectal cancer, colonic polyps, constipation, fecal impaction, diarrhea, hemorrhoids, hepatitis (A, B, C, D, and E), cirrhosis, fatty liver disease, liver cancer, cholecystitis, gallstones, pancreatitis, pancreatic cancer, food intolerances (e.g., lactose intolerance), parasitic infections (e.g., giardia or tapeworms), intestinal ischemia, gastroenteritis, or any other disease of or relating to the GI tract.

The capsule 10 can include any component configured to assist in the diagnosis or treatment of disease, for research, diagnosis, or any other suitable purpose, but some implementations are configured to deliver pharmaceuticals or other compounds, collect images or other data, provide or implant one or more sensors or other devices, collect one or more samples (e.g., tissue samples, gut microbiome samples, fecal samples, or other samples), target one or more specific areas of a patient, or otherwise assist (robotically, chemically, electrically, or otherwise) in diagnostic or treatment functions. In some cases, the capsule is configured to augment, assist in, or even replace more uncomfortable, expensive, invasive, dangerous, complicated, error-prone, or otherwise undesirable procedures, such as endoscopy, implant installation (e.g., stents or any other type of implant), gastric balloons, gastrectomy, vagotomy, nasogastric or gastrostomy tubes, bowel resection, colonoscopy, sigmoidoscopy, ostomy, biopsy, transplant, lithotripsy, cholecystectomy, pancreatectomy, paracentesis, ultrasound, CT scan, or any other diagnostic or treatment procedure of or relating to the GI tract. As an example, endoscopies and colonoscopies are often used for targeted drug administration, sample collection, data collection and inspection, or other diagnostic or treatment procedures, but they can be uncomfortable and expensive, and they often require a high level of expertise to perform correctly.

As examples of components that the capsule 10 can include to assist in diagnostic, treatment, or other procedures, some embodiments of the capsule include one or more housings 12, power sources 14, sensors 16, transmitters 18, or localization systems 20. In some cases, the localization system includes one or more anchors 22, strands 24, spools 26, or detectors 28. These and other components are discussed in additional detail below.

Where the capsule 10 includes one or more housings 12, the housing can include any component configured to contain one or more parts of the capsule. In some cases, one or more components of the capsule are positioned exterior to the housing (e.g., a sensor 16, an anchor 22, or another component), but in some cases most or all of the components are housed within the housing.

The housing 12 can be formed of any suitable material, including one or more of metal (including any suitable metal or metal alloy), plastic, carbon fiber, polymer material, glass, ceramic, wood, natural substance, synthetic material, pH-sensitive dissolvable material, or any other material. Indeed, in some embodiments, the housing includes one or more pH-sensitive dissolvable materials that are configured to dissolve upon exposure to one or more specific gastrointestinal conditions. Examples of such materials include one or more of: hydroxypropyl methylcellulose (HPMC), polyvinyl alcohol (PVA), carboxymethyl cellulose (CMC), methacrylic acid copolymers (including various Eudragit® formulations, such as Eudragit® L, Eudragit® S, Eudragit® FS, Eudragit® E, and Eudragit® RL/RS grades), cellulose acetate phthalate, hydroxypropyl methylcellulose phthalate, polyvinyl acetate phthalate, shellac, alginate compounds, pectin-based compounds, zein, chitosan, gelatin modified with one or more pH-sensitive cross-linkers, pH-sensitive hydrogels, acrylic-based polymers with pH-responsive side chains, modified starch derivatives with one or more acid-labile bonds, pH-sensitive polyelectrolyte complexes, carbomer-based polymers, or any combination thereof.

Where the housing 12 comprises one or more pH-sensitive materials, such materials can be configured to dissolve at any desired pH range. Indeed, in some embodiments, the pH-sensitive materials are configured to dissolve at specific pH ranges corresponding to different regions of the gastrointestinal tract, such as acidic conditions in the stomach, mildly acidic to neutral conditions in the duodenum, or neutral to basic conditions in the intestines-enabling targeted dissolution at desired locations.

In some embodiments, the housing 12 comprises multiple pH-sensitive materials with different dissolution thresholds that are combined in layers or mixtures to create staged dissolution profiles. In some such embodiments, the housing can incorporate these pH-sensitive materials as a complete housing material, as a coating on another housing material, as an intermediate layer between other materials, as a plug or barrier covering an aperture or window, as a matrix material containing other active components, or in any other suitable configuration that enables controlled dissolution based on pH conditions.

Although some embodiments of the housing 12 (or one or more portions thereof) are opaque, some embodiments are (or comprise one or more portions that are) translucent or transparent, such as to allow for a camera or another sensor 16 (which, in some cases, is contained within the housing) to see through the housing or otherwise collect data relating to the exterior of the capsule 10. In some embodiments, the housing (or one or more portions thereof) is radiotransparent to allow for a transmitter (or a receiver) to communicate with a device extemal to the capsule or external to the patient.

The housing 12 can be any suitable shape, but in some embodiments it is generally pill-shaped (e.g., cylindrical (such as flat-cylindrical or elongated cylindrical, in some cases with rounded ends), spherical, toroidal, or otherwise shaped). In some embodiments, the housing does not have sharp corners or edges that could cause damage to the patient's GI tract.

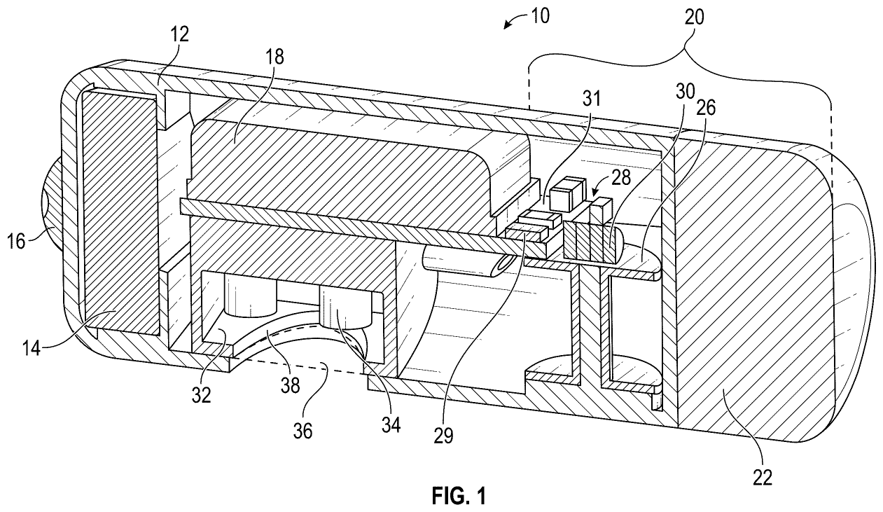

The housing 12 can also be any suitable size, such as any size that is reasonably ingestible, yet still large enough to house the necessary components. In some embodiments, the housing has a diameter of between 0.2 centimeters (cm) and 2 cm (and any subrange thereof, such as between 0.75 cm and 1.75 cm, between 0.9 cm and 1.5 cm, between 1 cm and 1.3 cm, or between 1.1 cm and 1.2 cm). In some embodiments, the housing has a length (e.g., a distance along a longitudinal axis) of between 0.3 cm and 5 cm (and any subrange thereof, such as between 1 cm and 3.5 cm, between 1.5 cm and 3.2 cm, between 2 cm and 3 cm, or between 2.5 cm and 3.5 cm). In some cases, the capsule has a size that is the same as a size of an already-FDA-approved capsule (such as the PillCam™ COLON 2). By way of non-limiting illustration, FIG. 1 shows an embodiment of a capsule 10 with a housing 12 that is substantially pill-shaped and that is configured to protect one or more interior components of the capsule from the capsule's external environment, the housing having a diameter of about 1.16 cm and a length of about 3.15 cm.

Where embodiments include one or more power sources 14, any reasonable power source configured to power one or more components of the capsule 10 can be used. That said, for purposes of size and efficiency, many embodiments of the power source include one or more batteries. In some cases, the power source is intended for a single use (e.g., the capsule is disposable), and in some cases, the power source is rechargeable (e.g., the capsule is reusable). By way of non-limiting illustration, FIG. 1 shows a capsule 10 that includes a built-in battery as a power source 14, the battery being fully contained within the housing 12.

According to some embodiments, however, the capsule 10 is configured to operate without a power source. In some such embodiments, the capsule can function in any suitable manner, including by utilizing natural peristaltic movements of the gastrointestinal tract. In such embodiments, the mechanical forces generated by intestinal contractions can be harnessed to drive one or more functional components of the capsule, such as rotating the spool to unspool the strand. Moreover, some implementations of the capsule include one or more mechanical energy capture mechanisms that are configured to convert peristaltic motion into rotational or linear movement, such as one or more ratchet mechanisms, clutch assemblies, unidirectional bearings, mechanical rectifiers, force-actuated gears, directionally-biased rollers, compliant mechanisms, spring-loaded components, cam-follower systems, or any other suitable mechanical components. In some cases, these mechanical systems can operate independently of any electrical power source, allowing the capsule to provide localization data through purely mechanical means (e.g., by leaving behind a physical trail of the strand (discussed below) that can be measured during post-passage examination) or in conjunction with a minimal power source (e.g., where mechanical systems handle primary operation functions but minimal power is used for data transmission). In some cases, this mechanical operation provides one or more features relating to reliability, longevity, and simplified construction, as the capsule's core localization functions are not necessarily dependent on battery life or electrical component failure.

Where the capsule 10 includes one or more sensors 16, any types of sensors can be included, such as optical sensors or cameras (i.e., visible light, infrared, or any other kind of optical sensors), pressure sensors, conductivity sensors, temperature sensors, pH sensors, proximity sensors, humidity sensors, audio or sonic sensors (microphones, ultrasound devices, etc.), accelerometers, gyroscopes, magnetometers, Hall sensors, sensors for detecting the presence of one or more particular compounds or any other suitable sensors. For example, some embodiments include a wireless endoscope, such that the location of an intestinal lesion or tumor can be ascertained. As another example, some embodiments include one or more electrodes 34 configured to measure conductivity, as discussed in more detail in a later portion of the specification.

In some cases, one or more sensors 16 are included on (e.g., mounted on or otherwise affixed to) an exterior of the housing 12 in order to more readily have access to the external environment of the capsule (where conditions to be sensed are often located). That said, in some embodiments, one or more sensors is included within an interior of the housing. In some such cases, the housing (or a portion of the housing) is configured to allow operation of the sensor from within the housing (e.g., by being transparent to allow for a camera to view the exterior), and in some such cases, the housing includes one or more apertures (e.g., a window 38, as discussed in more detail in a later portion of the specification) configured to allow the sensor to interact with the external environment of the capsule. In some embodiments with one or more apertures, the apertures are configured such that only the relevant sensor (or only the relevant portion of the relevant sensor) is exposed to the external environment, while other components within the housing are protected from the external environment (e.g., due to water-tight fittings or components or other protective elements of the housing). Some embodiments include only a single sensor, while some embodiments include multiple sensors (of the same or different types), such as 2, 3, 4, 5, 6, 7, 8, 9, 10, or more sensors. By way of non-limiting illustration, FIG. 1 shows a capsule 10 having a sensor 16 that includes a camera mounted on an exterior of the capsule's housing 12.

In some embodiments with a camera (or another optical sensor), a light source is included alongside the camera to enable the camera to better capture data about its surroundings. Similarly, one or more other sensors 16 can include other any other suitable auxiliary components configured to augment the functionality of such sensors (e.g., one or more light sources, ultrasound sources, amplifiers, signal boosters, reagents (for influencing reactions that might influence measurements), secondary sensors, or other auxiliary components).

According to some embodiments, the capsule 10 includes one or more passive mechanical sensing systems that require no electrical power to determine when a specific distance has been traversed. In such embodiments, when the strand 24 (discussed in more detail below) has completely unwound from the spool 26 (also discussed below), the mechanical resistance created by the fully extended strand prevents further forward movement of the capsule body through the GI tract, despite continued peristaltic forces. This mechanical “hard stop” can, in some embodiments, be used to trigger one or more therapeutic functions, such as releasing medication or collecting samples, at precise locations within the GI tract without requiring electronic sensors or power. For example, in some implementations, the resistance force created when the strand reaches its full extension can actuate a mechanical release mechanism, such as a pressure-sensitive latch, a tension-triggered spring, a force-activated valve, or any other suitable mechanical component configured to initiate a therapeutic action in response to the increased tensile load. By carefully selecting the length of the strand, the capsule 10 can be configured to automatically detect and respond to reaching specific target locations within the GI tract through purely mechanical means.

According to some embodiments, the capsule 10 includes one or more transmitters 18 and/or receivers. In this regard, the transmitter can include any component configured to transmit or assist in the transmission of one or more signals of any kind. For example, the transmitter can include one or more radio frequency (RF) transmitters (e.g., amplitude modulation (AM) transmitters, frequency modulation (FM) transmitters, frequency-shift keying (FSK) transmitters, orthogonal frequency-division multiplexing (OFDM) transmitters), ultrasonic transmitters, conductivity transmitters, capacitance-level transmitters, concentration transmitters, or any other suitable transmitters. By way of non-limiting illustration, some embodiments include one or more RF transmitters. Where one or more transmitters are used, the transmitters can be used to help determine the location of the capsule (as described in more detail below), but in some cases, the transmitters are simply used to transmit localization data (or other data) from the capsule (e.g., such that a practitioner can be apprised of the capsule's location within a patient, or of other data collected by the capsule). Additionally, where the capsule 10 comprises one or more receivers, the receiver can include any suitable type of receiver configured to receive or assist in the reception of one or more signals of any kind (i.e., any suitable receivers capable of receiving transmissions from any of the aforementioned transmitters). While use of a receiver can provide the capsule with any suitable function, in some embodiments, the receiver can help allow the capsule to provide or receive an externally confirmation signal to help the practitioner proceed with administration of a treatment after confirming location data or to allow a practitioner to determine how to proceed with, carry out, or to abort a treatment.

Although other non-invasive diagnostic or treatment devices may exist, it is often difficult to accurately and precisely localize such devices within a patient. Indeed, oftentimes only general localization is possible (e.g., generally in the area of the stomach, versus generally in the area of the colon), or highly uncomfortable, bulky, expensive, or otherwise inconvenient equipment is required (e.g., extensive sensor arrays external to the patient). Accordingly, some embodiments include one or more localization systems 20 configured to assist in the localization of the capsule 10 within the patient.

The localization system 20 can include any component suitable for assisting in the localization of the capsule 10. For example, some embodiments include one or more of the following: radiopaque or otherwise structured elements configured to show up on one or more types of imaging (e.g., X-ray, MRI, CT scan, ultrasound, PET scan, or other types of imaging); transmitters configured to emit one or more types of signals for information or tracking purposes; sensors configured to measure physiological conditions; magnets or diamagnetic elements configured to influence or react to a magnetic field; or other elements that can assist in positional tracking of one or more components of the capsule 10 within the gastrointestinal tract. Some embodiments include external equipment as well (e.g., for creating a magnetic field, generating ultrasound waves, detecting signals, capturing images, or otherwise assisting in the tracking of the capsule's position). Some embodiments implement one or more algorithms to increase the accuracy or precision of the tracking.

Some embodiments implement electromagnetic wave tracking. In some such embodiments, one or more receivers is placed external to the subject, with such receivers being configured to precisely measure one or more of a direction-of-arrival (DOA) and a time-of-arrival (TOA) RF signal sent between the capsule 10 and the receiver (e.g., by one or more transmitters 18). In some embodiments, algorithms (in some cases, very complex ones) are then used to determine the location of the signal origin within the capsule.

Some embodiments utilize magnetic field tracking. In some such embodiments, the capsule 10 includes one or more magnets 30, and the subject wears or is otherwise associated with an extemal array of sensors (e.g., magnetic Hall sensors) configured to detect and measure the magnetic field of the magnet internal to the capsule. In some embodiments, algorithms (again, in some cases, complex ones) are used to determine the location and orientation of the magnet, and therefore, the capsule.

Some embodiments implement optical tracking (or other sensory tracking). In some such cases, the capsule 10 includes one or more sensors 16 configured to implement one or more of feature recognition and vector quantization to map recognizable features within the intestinal lumen from frame to frame and track distance in this manner.

According to some embodiments, the localization system 20 of the capsule 10 is configured to measure a linear distance travelled by the capsule (e.g., using odometry or any other suitable method). The term “linear distance” as used here does not necessarily mean a distance in a straight line, but rather the distance of the path the capsule takes through the GI tract (e.g., if the path were straightened into a straight line and then measured). Although some implementations can utilize algorithms, external equipment, or other (potentially expensive) components as part of the localization process (as discussed above), some implementations of the capsule utilize one or more mechanical approaches to distance tracking, allowing them to avoid complex algorithms or expensive extemal equipment needed by some other localization methods. Further, some implementations of the capsule are able to determine localization independently of any input from external systems. In some cases, the systems and methods have less of a negative impact on the subject, as large sensor arrays around the subject's abdomen are not necessary. Moreover, some instances allow for a simplified data transmission process, as (in contrast to some localization techniques) a time and a direction of a signal (e.g., an RF signal or any other type of externally detectable signal) do not need to be precisely measured, since the signal itself can carry the localization data (as opposed to trying to use the signal to calculate the localization). In some embodiments, tracking is accomplished without utilizing electromagnetic wave tracking, magnetic field tracking, or optical tracking (although some embodiments use one or more of such methods as secondary localization techniques in addition to a primary localization mechanism). In some cases, the capsule is configured to provide localization data independently of any external device.

In accordance with some embodiments, the localization system 20 is configured to determine a location of the capsule 10 within the GI tract of a patient via odometry. In some cases, the location of the capsule is continuously or intermittently tracked, along with a linear distance travelled by the capsule (e.g., using odometry). In some cases, this is accomplished by including (e.g., with the capsule) one or more anchors 22 configured to attach, lodge, or otherwise anchor in or associate with a specific location in the GI tract (or even another location, such as external to the subject's body). Some embodiments of the capsule are then configured to determine a distance that the body of the capsule travels with respect to the anchor (which has, in some cases, at least temporarily anchored to one or more structures and thereby halted its journey through the GI tract).

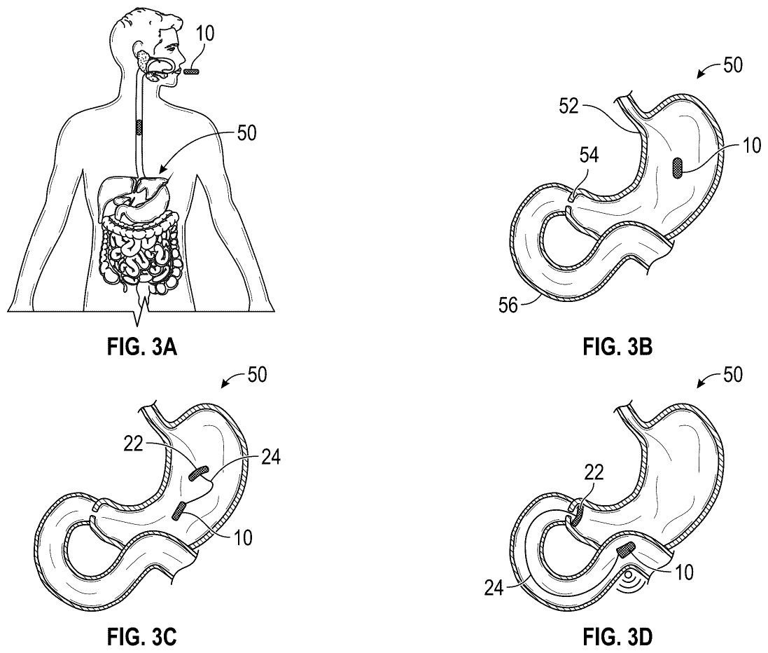

In some embodiments, the anchor remains attached to the body of the capsule via one or more strands 24. By way of non-limiting illustration, FIGS. 3A-3D show an example of how an odometry system of the capsule 10 may be used to track a distance traveled by the capsule and provide useful localization data. In FIG. 3A, a capsule 10 enters the GI tract 50 of a patient through ingestion. FIG. 3B shows the capsule in the patient's stomach 52, not yet having passed through the pyloric valve 54 into the duodenum 56. FIG. 3C shows the anchor 22 dissociating from the body of the capsule 10 in the stomach, the anchor being attached to the body of the capsule via a strand 24. In FIG. 3D, the anchor 22 becomes lodged in place (not passing through the pyloric valve, such as due to its size, shape, or other characteristics), while the body of the capsule 10 continues through the duodenum and down the GI tract (while still being tethered to the anchor via the strand 24.

Where the capsule 10 includes an anchor 22, the anchor can include any component configured to anchor to any portion of the GI tract, including any valve therein, any tissue or lining, any structure or substructure, or any other suitable anchor point (e.g., the lower esophageal sphincter (LES), the pyloric valve, the ileocecal valve, a tooth, the tongue, a cheek, a lip, any suitable exterior anatomy, or any other suitable fixed anatomical feature or other suitable anchor point). For example, some embodiments of the anchor are configured to anchor to or associate with (e.g., be too large or incorrectly shaped to pass through) the pyloric valve.

According to some embodiments, the anchor 22 is configured to deploy. In some cases, deployment includes one or more of: (a) the anchor dissociating from the body of the capsule 10; and (b) the anchor expanding, changing shape, releasing or exposing a catch, or otherwise adopting an anchoring configuration. For example, in some cases, the anchor is relatively small and attached to the body of the capsule 10 for easy ingestion, then the anchor deploys by dissociating from the capsule and expanding, thereby lodging in place while allowing the body of the capsule to continue down the GI tract.

The anchor 22 can be configured to dissociate from the body of the capsule 10 in any suitable manner. For example, in some embodiments, the capsule is configured to trigger release of the anchor upon sensing certain gastric conditions (e.g., when a sensor detects a certain pH). That said, in some embodiments, the capsule includes one or more enteric coatings configured to dissolve upon presence of certain gastric conditions, thereby triggering release of the anchor.

Where the capsule 10 includes one or more enteric coating (used as the housing 12, used to cover a portion of the anchor 22, or otherwise associated with the capsule), the enteric coating can include any suitable coating configured to release the anchor at any desirable point within the GI tract (e.g., in the stomach, in the small intestine, or at any other suitable point). In some cases, the enteric coating includes one or more enteric polymers (configured to dissolve at a specific point along the GI tract, such as in response to certain gastric conditions). In various embodiments, enteric coatings can include ethyl cellulose, cellulose acetate phthalate, hypromellose phthalate, cellulose butyrate phthalate, cellulose hydrogen phthalate, hydroxypropyl methylcellulose phthalate, cellulose proprionate phthalate, polyvinyl acetate phthalate, hydroxypropyl methylcellulose acetate succinate, dioxypropyl methylcellulose succinate, hydroxypropyl methylcellulose trimellitate, triethylcitrate, polyvinyl acetate phthalate, methylmethacrylate esters, shellac, any other pharmaceutically acceptable enteric materials (whether fatty acids, polyacids, waxes, plastics, plant fibers, gelatins, solutions of film resins, or any other suitable materials), any materials used for the housing (as described herein), or any other enteric coating materials. In some cases, the enteric coating is configured to dissolve upon reaching the stomach of the subject (although in some embodiments, the enteric coating is configured to keep the anchor 22 associated with the body of the capsule 10 until a different target area is reached, such as the jejunum of the small intestine or another area in the GI tract).

According to some embodiments, the anchor 22 is configured to expand or otherwise act as an anchor upon dissociation from the body of the capsule 10 (for example, in some cases, the enteric coating prevents the anchor from expanding, then once the enteric coating has dissolved, the anchor is allowed to expand). The anchor can be configured to expand or move to an engagement configuration in any suitable manner, such as by including one or more compliant mechanisms (e.g., springs or other compliant materials configured to naturally expand or move if allowed), absorbent materials (e.g., configured to expand upon absorption of stomach acid, water, or other GI tract content), inflatable components (e.g., configured to inflate upon dissociation from the body of the capsule, such as by trigging a gas-producing reaction inside a balloon-like element, releasing gas from a pressurized chamber into a balloon-like element, drawing in fluid via osmosis or active diffusion, or otherwise inflate).

Where the anchor 22 includes one or more compliant mechanisms, the compliant mechanisms can include any suitable mechanisms configured to cause the anchor to expand as desired. For example, some embodiments include one or more springs or resilient members (beginning in a compressed configuration and configured to move to an uncompressed configuration to deploy the anchor), plates or sheets (which may be folded and configured to unfold to expand the anchor when allowed), or any other components configured to cause the anchor to expand.

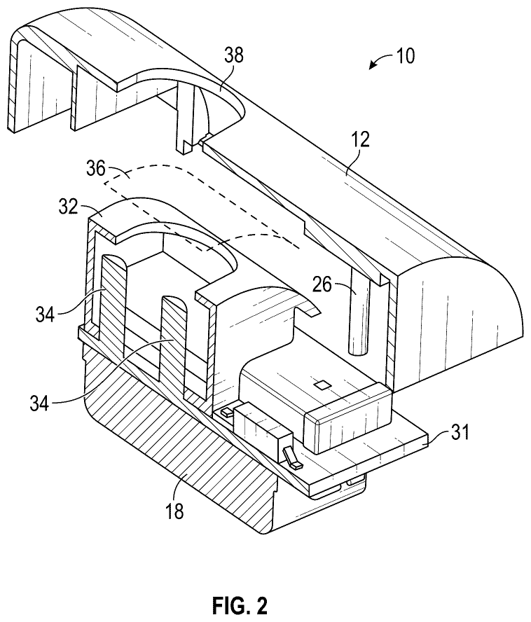

The anchor 22 can be configured to expand any suitable amount. For example, in some embodiments the anchor is configured to expand to between 1.1 and 30 times (1.1×-30×) its original size (or any suitable subrange thereof, such as between 1.7× and 4×, 1.9× and 3.5×, 2× and 3×, or any other suitable subrange). In some cases, the anchor is configured to expand to a certain size (e.g., large enough to become lodged in a specific portion of the GI tract, to get caught on a valve (e.g., the pyloric valve), or to otherwise anchor itself in place as designed). By way of non-limiting illustration, FIG. 2 shows an anchor 22 with built-in compliant mechanisms configured to cause a diameter of the anchor to expand from about 1.6 cm to about 2.5 cm. Thus, the anchor (in accordance with the embodiment shown in FIG. 2) or one or more portions thereof, such as a diameter, is configured to expand to larger than a diameter of the pyloric valve (which is generally about 1.3 cm-2 cm) to ensure that the anchor does not pass through the pyloric valve (e.g., to retain the anchor within the stomach). According to some embodiments, the expansion is configured to take place along at least two axes, and in some cases all three axes (e.g., an X, a Y, and a Z axis). While the anchor can expand to move to any suitable shape (e.g., a bar shape, an X-shape, an I-shape, disc shape, a plate shape, a grate shape, or any other suitable shape), in some embodiments, the anchor is configured to allow fluids to flow past it, once the anchor is deployed.

According to some embodiments, the anchor 22 is configured to break down (e.g., in gastric or other physiological conditions) or otherwise un-deploy after a certain amount of time such that it no longer acts as an anchor and can safely pass through the remainder of the GI tract. For example, in some cases, the anchor includes one or more components configured to dissolve over time such that the anchor falls apart and passes through the GI tract. That said, some embodiments include a timer, a remote dissociation mechanism (e.g., an extemal switch), or another mechanism configured to cause the anchor to break down, shrink, collapse, or otherwise un-deploy (i.e., stop its anchoring functions such that it can pass through the GI tract). In some cases, the anchor is configured to release after a time interval of between 2 hours and 3 weeks (or any suitable subrange thereof, such as between 12 hours and 7 days, 1 day and 2 days, 25 hours and 100 hours, 30 hours and 70 hours, 55 hours and 65 hours, or any other suitable subrange). In some cases, the anchor is configured to remain in place for at least 25 hours to 35 hours, as it typically takes about 30 hours for the body portion of the capsule 10 to make its way through the remainder of the GI tract and collect the necessary data.

Where the capsule 10 includes one or more strands 24, the strand can include any type of suture, string, thread, chain, filament, fiber, cable, or other elongated component capable of coupling the anchor 22 to the body of the capsule. For example, some embodiments of the strand include a suture, such as a dissolvable suture or other dissolvable or bioabsorbable material to ensure that the strand does not get caught in the GI tract or cause future complications due to its presence (and that the subject does not need to pass the strand while it remains in strand form, which could be uncomfortable). Where a dissolvable suture is used, any suitable gauge or type of suture can be used, but some embodiments include a 7-0 gauge suture.

The strand 24 can be attached to the anchor 22 in any suitable manner, such as via one or more one or more nails, screws, bolts, staples, eyelets, magnets, hook-and-loop fasteners, adhesives, welds, interference fits, friction fits, mechanical engagements, catches, tongue-and-groove connections, snaps, ties, rivets, stakes, wire ties, integral formation, or any other manner of attachment.

According to some embodiments, the strand 24 is configured to pass through a passage formed in the housing 12 of the capsule 10. In some cases, this passage is small, such that it can only fit the strand through, or the passage is otherwise water-tight to prevent liquid or other material from entering the capsule through the passage.

In some embodiments, the strand 24 is configured to associate with (e.g., be stored in or around) a spool 26. Where embodiments include a spool, the spool can include any component for releasably storing the strand (e.g., in a wound, coiled, folded, compressed, or otherwise stored state). By way of non-limiting illustration, FIG. 1 shows a bobbin-like spool 26 disposed near an anchor end (an end nearest the anchor 22) of a body of the capsule 10, the spool being configured to rotate to unwind, letting out the strand length by length (through a small passage) as the capsule body separates from the anchor.

According to some embodiments, the capsule 10 includes one or more detectors 28. The detector can include any component configured to detect, count, quantify, or otherwise measure the amount (e.g., the length) of the strand 24 that is unspooled from the spool 26. For example, some embodiments of the detector include one or more of any of the following: optical or other sensors configured to detect markings on the strand 24 (thereby determining a length of the strand that is unspooled based on the number of markings detected); optical or mechanical sensors to measure a diameter (or other characteristic) of remaining strand on the spool; scales or other detectors configured to evaluate the weight or mass of the spool (thereby determining how much of the strand has been unspooled due to the decrease in mass of the spool); rotation counters configured to keep track of the rotations of the spool (to thereby determine how much of the strand has unwound from the spool based on the spool's rotations); or any other measurement mechanism configured to assist in ascertaining the length of the strand that has unspooled.

Where the detector 28 is configured to measure the rotations of the spool 26 as the strand 24 unwinds from the spool, the detector can be configured to do so in any suitable manner. For example, the spool can include: one or more markings configured to be detected by an optical or other sensor; one or more protrusions or other features configured to push a button or otherwise increment a counter as the spool rotates; one or more Hall sensors 29 configured to detect a rotating magnetic field of one or more magnets 30 coupled to the spool, thereby determining the number of spool rotations based on the number of magnetic field rotations; or any other rotation tallying mechanism. In some cases, the capsule 10 (e.g., a capsule that determines its distance mechanically) is optionally configured to detect or identify when the strand has been entirely unwound to indicate that the capsule is at a known position with respect to the anchor. While this can be accomplished in any suitable manner, in some cases one or more sensors, switches, or mechanisms are configured to identify when the strand has been fully deployed.

Where the detector 28 includes one or more Hall sensors 29, the Hall sensors can include any sensors configured to detect and measure a magnetic field. In some cases, the Hall sensor is configured to sense a dynamic magnetic field produced by one or more rotating magnets 30 (e.g., a magnet mounted on the spool 26). The magnets themselves can be mounted on the spool in any suitable manner (e.g., on top of it, on bottom, on the side) that generates a magnetic field that varies (as detectable by the Hall sensor) when the spool rotates. For example, in some cases, the magnets are oriented such that the north and south poles of the magnets are substantially parallel with a tangent of the spool (e.g., a longitudinal axis of the magnets that passes through both poles is substantially aligned with a diameter of the spool). Any suitable number of magnets can be used (e.g., 1, 2, 3, 4, 5, 6, or any other practical number). By way of non-limiting illustration, FIG. 1 shows a linear magnetic Hall sensor 29 (e.g., Digi-Key, PN: RR1 12-1G42-532) configured to detect a rotating magnetic field generated by four magnets 30 (e.g., Amazing Magnets, PN: D021-063) mounted on top of a spool 26.

While the Hall sensor 29 can be set up to detect the magnetic field in any suitable manner, in some embodiments it is integrated with a printed circuit board (PCB) 31. In this regard, some embodiments include a PCB, which in some cases is electronically coupled with the detector 28 (e.g., the Hall sensor 29). The PCB can include or couple to any component useful for the electronic operation of the capsule 10. For example, in some embodiments, the PCB is electronically coupled to the power source 14, which can then be used to provide power to any other component coupled to the PCB. In some embodiments, the transmitter 18 is coupled to the PCB. In some embodiments, the PCB includes one or more processors configured to assist with the operation of the capsule 10 (or any portion thereof).

According to some embodiments, the PCB includes one or more controllers configured to interpret data from the detector 28. For example, in some embodiments, the controller is configured to read the magnetic field (as detected by the Hall sensor 29) as a percentage of the supply voltage (Vcc), with no magnetic field present being 50% of the Vcc. In some cases, two voltage thresholds, one above 50% and one below 50%, are set to detect when the north or south poles of the magnets are facing the Hall sensor. As the magnets rotate and the poles facing the Hall sensor alternate, the controller is configured to sense the fluctuations in the Vcc, interpreting these as half-revolutions of the spool 26. The revolution count can then be converted to a distance using a distance conversion model (as discussed in more detail later) relying on the dynamic circumference of the threaded spool. By way of non-limiting illustration, some embodiments include a controller in the form of a microcontroller, such as a PIC12F1572 microcontroller.

According to some embodiments, the capsule 10 includes one or more compartments 32. The compartment can include any cavity or space for containing additional capsule components, medication, testing equipment, sensors 16, or any other item or material useful to be contained within the capsule. The compartment can also be used for collecting samples or otherwise obtaining material from the GI tract of the patient for delivery to the exterior of the patient (e.g., following defecation). By way of non-limiting illustration, FIGS. 1-2 show capsules 10 having compartments 32 that house sensors in the form of electrodes 34.

In some embodiments with one or more compartments 32, one or more of such compartments is separate from other portions of the capsule's 10 interior. For example, in some cases, the compartment is water-tight such that fluid in the compartment (e.g., medicine, gastric fluid, or other fluid) cannot contact the PCB 31, the power source 14, or other sensitive portions of the capsule's interior.

In some embodiments, the capsule 10 includes one or more windows 38. The window can include any aperture, passage, hole, or other opening configured to allow one or more items or materials to enter or exit the capsule (e.g., intestinal fluid, light, medication, samples, the strand 24, or anything else). The window can have any shape (e.g., circular, semi-circular, triangular, square, rectangular, trapezoidal, pentagonal, hexagonal, star-shaped, T-shaped, polygonal, or any other regular or irregular shape). The window can also be positioned on any portion of the capsule (e.g., positioned at an end near the anchor 22 to allow the strand to exit, positioned near a compartment 32 to allow the compartment to have fluid communication with the GI tract, positioned near a sensor 16, such as a camera positioned on an interior of the capsule to allow interior sensor to have a view of the exterior of the capsule, or in any other suitable position).

According to some embodiments with a window 38, the window is covered with one or more barriers 36. The barrier can include any suitable barrier, such as a pane, a membrane, a film, a sheet, or any other barrier. In some embodiments, the barrier is configured to prevent fluid or other materials from passing through the window (e.g., the barrier can act like a pane of glass in a traditional window, selectively allowing light (or other desirable materials, such as medication) through, while keeping intestinal fluid out of the capsule). For example, in some cases, a transparent barrier allows a camera to see out through a window without being subject to potentially harmful gastric or other GI tract conditions.

Some embodiments of the barrier 36 are configured to selectively dissolve. For example, in some cases, the barrier is configured to withstand gastric conditions, then dissolve in the small intestine (or another desirable location) to collect a sample, trigger transmission, release a medication, or otherwise effectuate a change in the capsule 10. By way of non-limiting illustration, FIG. 2 (as discussed in more detail in example 4) shows a capsule that uses a barrier 36 in the form of a dissolvable film configured to selectively trigger transmission of data upon dissolution. While the barrier can comprise any material, in some embodiments, it comprises one or more materials used for the housing, a one-way permeable material, or any other suitable material.

According to some embodiments, the capsule 10 is configured not only to track the capsule's location or linear distance travelled, but also to localize to a specific location (e.g., to travel to the mid-large intestine and then stop its forward progress through the GI tract). This can be done in any suitable manner, but in some cases it is done by having a strand 24 of a specific length, such that when the spool 26 is fully unwound, the capsule cannot make additional forward progress (e.g., one end of the strand is attached to the anchor 22, and the other end of the strand is attached to the spool or another part of the capsule body).

The components discussed herein can be formed of any suitable materials and in any suitable manner. For example, any suitable component of the capsule 10 is formed of or includes one or more of the following: metal, glass, plastic, carbon fiber, polymer material, leather, organic material (e.g., natural or artificial skin, muscle, bone, or other tissue), wood, cardboard, paper, nylon, fabric, natural substances, synthetic materials, pH-sensitive dissolvable materials, bioabsorbable materials, or any other suitable material. In some cases, a specific material or a material with a specific attribute is required, such as magnetism, dissolvability, bioabsorbability, or another particular trait (as discussed above), and in such cases, the material is selected to have the necessary or desired attributes.

The components of the capsule 10 can also be formed in any suitable manner, such as via one or more of casting (e.g., continuous casting, die casting, mold casting, resin casting, sand casting, or other casting), molding (e.g., metallurgy, compaction, injection molding, or other molding), forming (e.g., forging, extrusion, pressing, or other forming), machining (e.g., milling, turning, drilling, or other machining), joining (e.g., welding or other joining), additive manufacturing (e.g., 3D printing, deposition, layered manufacturing, or other additive manufacturing), subtractive manufacturing, machining, extrusion, carbine, stamping, punching, pressing, or otherwise. By way of non-limiting illustration, some embodiments of the capsule include a 3D-printed housing 12.

According to some embodiments, a method of providing a capsule 10 is provided. In some embodiments, the method includes obtaining the capsule, which can include forming, manufacturing, purchasing, or otherwise obtaining any of the components of the capsule as discussed herein, and assembling them, modifying them, configuring them to form the capsule, and otherwise obtaining the capsule. By way of non-limiting illustration, some embodiments include one or more of: forming a housing 12; installing (e.g., electronically coupling to a PCB 31) a power source 14; installing a sensor 16; installing a transmitter 18; installing a localization system 20; coupling an anchor 22 to a body of the capsule 10; coupling the anchor to the body via a strand 24; spooling the strand on a spool 26; installing a detector 28 (e.g., including a Hall sensor 29 and a magnet 30); or including any other components in any configuration discussed herein.

As discussed above, according to some embodiments of the capsule 10, the capsule includes one or more spools 26 wound with one or more strands 24 (e.g., of a dissolvable suture thread). In some cases, the spool is configured to rotate as the capsule traverses a portion of the GI tract, thereby leaving behind a trail of strand. In various implementations, the rotations of the spool are tracked by the capsule and mapped to a linear distance traveled by the capsule. Some implementations of the capsule utilizing such a tracking method are able to accurately track distances of up to 10.5 meters (m) or more (and, of course, less), with very low error margins. That said, in some cases, smooth, efficient, and consistent winding of the spool is important to ensure that the strand does not tangle and that distances can be mapped reliably. Accordingly, some embodiments include one or more methods for winding the spool (e.g., as part of the method for providing the capsule or as a stand-alone method).

In some cases, the method for winding the spool 26 includes utilizing a custom setup of fixtures in connection with a bobbin winder (e.g., a Singer® Tradition sewing machine, such as Model 2277 with a bobbin winding function). In some cases, the method includes obtaining (e.g., designing, forming, manufacturing, or otherwise obtaining) a custom adapting fixture configured to allow the spool to be inserted into the bobbin winding mechanism. According to some embodiments, activating the bobbin winding mechanism (e.g., by pressing a foot pedal or otherwise), the suture (or other strand) runs through a tensioner, over a leveling fixture, and onto the rotating spool. In some cases, the leveling fixture raises the level of the suture (or other strand) as it is wound around the spool, allowing for a more even winding along the entire length of the spool.

As mentioned above, some embodiments of the spool 26 are configured to be wound with up to 10.5 m of suture or other strand (e.g., allowing the capsule 10 to measure up to 10.5 m of intestinal length), but in some cases the amount of strand wound on the spool is patient-specific. Accordingly, in some embodiments the method includes measuring or estimating a length of a portion of the patient's GI tract and winding an amount of strand on the spool that corresponds with or is shorter than such length. In this regard, a patient-specific amount of strand can prevent the capsule from being passed during defecation while the strand is still attached (which could be unpleasant for the patient). In accordance with the foregoing, some embodiments of the spool are wound with between 1 m (such a short length could be necessary in a patient with abnormally short intestines, such as patients who have had bowel resections) and 15 m of strand, or any subrange thereof (e.g., between 6 m and 13 m, 8 m and 12 m, 9 m and 11 m, or any other suitable length).

According to some embodiments, the method of providing the capsule 10 includes utilizing a distance conversion model to calculate the distance traveled by the capsule based on the rotations of the spool 26. In this regard, in some embodiments, to accurately calculate the distance traveled by the capsule, the diameter of the strand 24 as wound on the spool must be known, but as more suture is unspooled, the diameter (and resulting circumference) of the strand wound on the spool decreases. Thus, to account for this, some embodiments implement a mathematical model to convert the revolution count (e.g., as measured by the controller based on data from the Hall sensor 29) into a linear distance traveled by the capsule. Example 1 (below) shows an example of experimental results connected with formulating a distance conversion model (accordingly, some embodiments of the method include implementing any of the actions or utilizing any of the components as discussed in Example 1, or in any of the other examples).

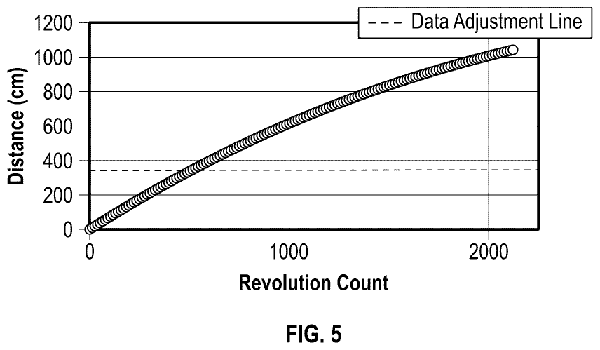

According to some embodiments, the method includes using the distance conversion model as a master model for any length of strand 24 wound onto the spool 26. In some cases, the length of strand wound on the spool affects the diameter of the spool (or rather, the diameter of the strand wound on the spool), with more strand leading to a larger diameter that decreases as the strand unwinds. In some embodiments, a mathematical model (as discussed in connection with Example 1) is altered to provide an accurate model for any length of the strand on the spool (as discussed in connection with Example 2) by taking into consideration the change in the spool's initial outer diameter (for example, the master model's data can be removed up until the remaining data matches the length of the strand). By way of non-limiting illustration, FIG. 5 shows data from a master model with 10.5 m of strand, but to create a model for a 7-m strand spool, the data below the horizontal, dashed data adjustment line (the line is located at distance=3.5 m, as 10.5 m minus 3.5 m equals 7 m) is removed. A new best fit quadratic function is fit to the data above the data adjustment line, becoming the model for that length of spool. By moving the data adjustment line up or down and re-fitting curves to the resulting data, models can be created for virtually any length of strand wound onto the spool.

The described systems and methods can be modified in any suitable manner. For instance, in some embodiments, the roles of the anchor 22 and the body of the capsule 10 are reversed, such that the spool 26 and its associated components (e.g., the detector 28) are housed within the anchor portion (e.g., they are configured to remain within the stomach or another anchor point of the GI tract), while the body of the capsule unwinds the strand by virtue of its travel through the GI tract.

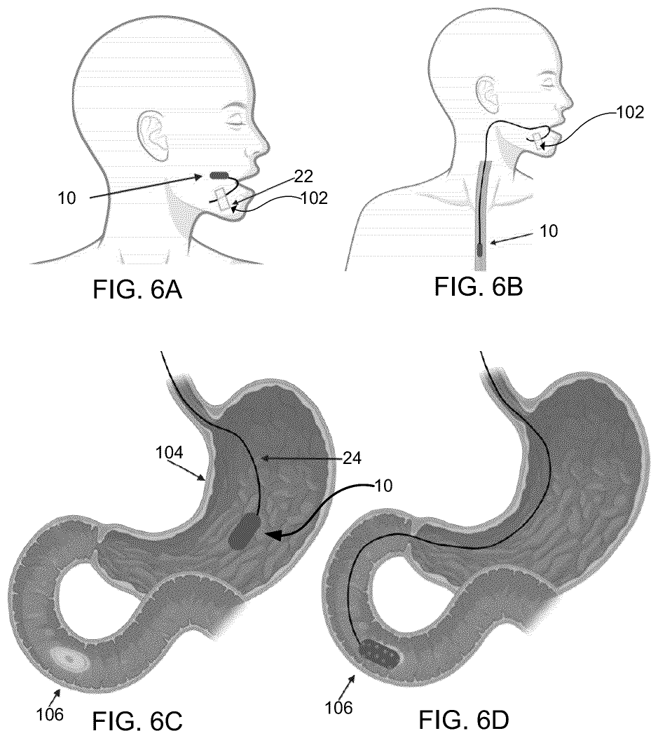

As another example of a suitable modification, instead of being configured to be anchored relatively deep within the GI tract, in some embodiments, the strand 24 is configured to be anchored (as mentioned earlier) in the subject's mouth, to one or more of the subject's teeth, to the subject's tongue, to the subject's face (e.g., external cheek, chin, nose, etc.), or to any other suitable location. Thus, in some embodiments, the anchor 22 need not deploy after the capsule 10 has been swallowed. Indeed, in some embodiments, the anchor comprises one or more tethers, knots, loops, adhesives, pieces of tape, clamps, frictional engagements, mechanical engagements, or any other suitable component that is capable of anchoring the strand in, around, or outside of a subject's mouth. By way of non-limiting illustration, FIG. 6A-6B show some embodiments in which the strand 24 is coupled to a patient's cheek via an adhesive (e.g., a piece of tape 102) that serves as the anchor 22, such that the capsule 10 is able to be ingested into the subject's GI track (e.g., moved down into or past the stomach 104) while determining the location of the capsule 10. Indeed, as shown in FIGS. 6C-6D, in some embodiments, the capsule 10 (and the described systems and methods) allow for a practitioner to use the capsule to identify, locate, observe, treat, biopsy, or otherwise address a location of a point of interest (e.g., a lesion 106).

As yet another example of a suitable modification, in some embodiments, the strand 24 comprises a thread coil (e.g., a coil that is not wound on a spool 26). In some such embodiments, as the capsule moves through the GI tract, the uncoiling thread moves through or otherwise engages one or more rollers that comprise, or are configured to work with, any suitable type of sensor (i.e., any suitable sensor described herein, such as one or more Hall sensors) to permit odometry of the uncoiling thread.

In addition to the aforementioned features, the described systems and methods can include any other suitable feature. According to many traditional localization methods, such methods are able only to provide coordinates of a single location within 3D space (or in some cases, a series of disjointed, unconnected 3D coordinates). In contrast, some embodiments of the capsule 10 are configured to provide a continuously mapped path through the intestinal tract. In other words, some embodiments are able to provide measurements and information regarding the accumulated distance traveled by the capsule, as well as the relative position of the capsule within any particular 3D region (without needing to rely on electromagnetic, magnetic, or other external sensors that are subject to change during bodily movement and natural intestinal shifting, and without needing to rely on complex algorithms). In some cases, traditional methods (e.g., which are often required to implement complex algorithms) often result in 5 millimeters (mm)-70 mm of static position error (or more) due to the complex nature of signal tracking through the various layers of human tissue, as well as the lack of precise linear distance data of the capsule traveling along the GI tract. Moreover, due to the intestinal lumen's winding nature and lack of defining features (in some parts), tracking errors in optical tracking are also common. Thus, the odometry system found in some embodiments of the present systems and methods is extremely useful for providing improved localization data (either used alone or in combination with one or more additional tracking methods).

Some embodiments have advantages over some other tracking systems in that they are not directionally-dependent (for example, in some cases, the capsule 10 can enter through the pyloric valve in any orientation and still function properly, unlike some previous devices with expandable wheel systems). Moreover, some embodiments are not reliant on wheels or other rotating components exposed to the environment, which can, in some cases, get stuck in mucosal lining or otherwise be inhibited, thereby contributing to large-accumulated error along the length of the GI tract.

Initial testing of the systems and methods described herein shows promising levels of accuracy, autonomous capability, simplicity, and potential robustness to the tortuous and slippery GI environment, which are superior features to many current localization techniques.

### EXAMPLES

### Example 1

A partially assembled capsule including the PCB (with attached transmitter), odometer assembly (e.g., detector, Hall sensor, magnets), and a power supply were mounted to a workbench next to a tape measure. The odometer spool was wound with 10.5 m of suture. The RF transmitter on the PCB transmitted data to a receiving module configured to display a single number representing the number of revolutions (beginning at 0) measured by the Hall sensor. The free end of the suture was held and pulled out of the capsule in 5 cm increments with the revolution count recorded by the device at each increment. This was repeated once more with an identical parameter set. The data from these two trials was averaged and plotted (as shown in FIG. 5). A best-fit quadratic function was fit to the data to provide a model converting revolution count into distance.

To convert the number of spool rotations sensed by the internal mechatronic system into a distance, a model was created by fitting a second order polynomial curve to empirically gathered data. A master model was created for the longest length of suture potentially wound on the spool (in the particular experiment in question, this was 10.5 m). Subsequent models for shorter lengths of strand were also created. Models for various lengths of strands are shown in Table 1, in which D stands for distance in centimeters and C stands for spool revolution count:

TABLE 1
Distance Model

| Length of Spool (m) | Model |
|---|---|
| 10.4 | D = −0.000113*C² + 0.7285989*C + 2.3353178 |
| (Master Model) | |
| 8 | D = −0.000112*C² + 0.6504227*C − 0.854752 |
| 6 | D = −0.00011*C² + 0.5724122*C + 2.2749654 |
| 4 | D = −0.000108*C² + 0.4899129*C + 0.02244957 |

### Example 2

To ensure the validity of altered models created for shorter lengths of suture, the experiment in Example 1 was repeated with various shorter lengths of suture. Multiple trials with different lengths of suture were performed. The resulting error between the distance predicted by the model and the actual known distance was recorded for each 5 cm incremental data point. The error between the distance predicted by the model and the actual distance (as measured during experimentation) was calculated. The error was averaged over each of the data points gathered at 5 cm increments during experimentation. Table 2 shows the average error and standard deviation for each trial as well as the maximum error seen in each trial. The average error across all trials was 7.33 mm±5.34 mm.

TABLE 2
Localization Error for Distance Modeling Trials

| Model | Average Error (cm) | Maximum Error (cm) |
|---|---|---|
| Master | 0.86 ± 0.65 | 3.09 |
| 7.7 m Trial 1 | 1.91 ± 1.88 | 5.56 |
| 7.7 m Trial 2 | 1.12 ± 0.93 | 3.06 |
| 4.75 m Trial 1 | 0.96 ± 0.58 | 2.59 |
| 4.75 m Trial 2 | 0.37 ± 0.29 | 1.63 |
| 3.2 m Trial 1 | 0.31 ± 0.30 | 1.50 |
| 3.2 m Trial 2 | 0.33 ± 0.23 | 1.09 |

### Example 3

To ensure location data could be sent reliably from the capsule to an external receiver, an experiment was designed to optimize circuitry allowing transmission through human tissue. For wireless transmission of data, the Linx 433 MHz RF transmitter and receiver modules were used. This specific frequency of signal was chosen as lower frequencies of RF signals tend to attenuate less and are generally absorbed less by human tissue. The Linx txm-433-lr transmitter module is a compact, self-contained RF transmitter requiring only a data input and an antenna as external connections. Two bits of data representing the count of spool revolutions were sent to the data port of the transmitter every 2 seconds, which was sent wirelessly to the Linx rsm-433-lr receiver module. The receiver was connected to an external Arduino Uno which received the data and displayed it on the serial monitor for observation. The receiver module was also programmed to output a value representing the strength of the signal. This signal strength value was mapped to values between 0 and 100, representing a signal strength percentage which is displayed with each data package received. Note that in this experiment, external electronics are not needed for feedback to the device (i.e., it can operate autonomously without external hardware). In this case external hardware was used for device validation only.

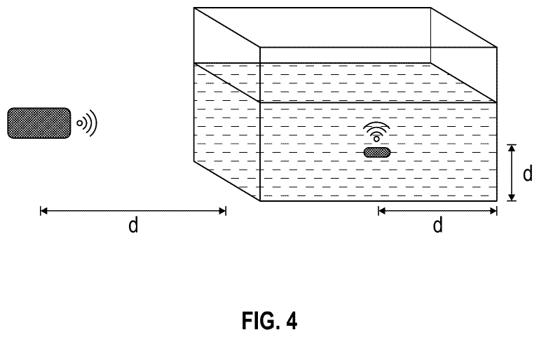

Due to the limited size of the capsule, power management becomes a limiting factor. This requires, in some cases, that transmission power is balanced with current draw. A resistor can be used in conjunction with the RF transmitter module to vary the transmission power, resulting in different current draws from the battery. To determine the lowest current draw that would allow reliable data transmission from inside of the intestinal tract, the transmitter was sealed inside of the capsule and placed inside of a plastic container with 30 cm of water on all sides of the capsule (as shown in FIG. 4), which is generally a reliable test to simulate data transmission through tissue. The receiver was placed at the edge of the container and gradually moved farther away from the edge until the signal was lost, with that final distance being defined as the maximum reliable transmission distance (d, in FIG. 4). Four power-limiting resistors (with the following values: 200 Ohms, 510 Ohms, 1 k Ohms, and 2 k Ohms) were each tested in this environment. The current draw from the device with each of these resistors was also recorded to find the best trade-off between current draw and transmission distance. The voltage drop across a resistor in series with Vcc was measured and used to calculate both idle current draw (not transmitting) and the active current draw while transmitting.

A summary of the RF transmission testing results can be seen in Table 3. For each of the current-limiting resistors, the idle (no data transmission) current draw, transmitting current draw, and maximum reliable transmission distance (see FIG. 4) are presented. As expected, both the idle and transmitting current draws increased as the resistance decreased. However, this did not directly correlate to transmission reliability. The maximum reliable transmission distance was the highest with the 510 Ohm resistor, unexpectedly decreasing with the 200 Ohm resistor. For this reason, the 510 Ohm resistor was selected for the final design (according to some embodiments). With this resistance, a maximum transmission distance of 0.76 m was observed, in addition to the transmission through 30 cm of water. This simulates transmission through the body with 0.76 m of buffer for the external receiver before the signal would be lost.

TABLE 3
RF Transmission Results

| | Idle | Transmitting | Maximum |
|---|---|---|---|
| Resistor | Current | Current | Reliable |
| Value | Draw | Draw | Transmission |
| (Ohms) | (μA) | (mA) | Distance (m) |
| 200 | 72.6 | 7.31 | 0.25 |
| 510 | 67.5 | 6.78 | 0.76 |
| 100 | 50.8 | 6.38 | 0.53 |
| 2000 | 25 | 5.81 | N/A |

### Example 4

To observe the odometer autolocalization system employed in a realistic medical capsule environment, it was implemented into a custom-designed capsule 10 that works in conjunction with polymer films sometimes used in medical capsule technology. The device detects when the film has dissolved and begins transmitting the distance traveled by the capsule to the user, informing the research team about the dissolution profile of the film. To achieve this, a capsule was designed with a hole in the outer shell (e.g., a window 38) that exposes the film (e.g., a barrier 36) to the GI environment. The film was sandwiched between this hole in the capsule housing and an inner cavity (e.g., a compartment 32) in which two copper electrodes (e.g., electrodes 34) are located (see FIG. 2). These electrodes are each connected to a pin on a microcontroller (e.g., the controller on the PCB 31) with one set as a high output and the other as an input with a pull-down resistor to ensure an initial low value reading. Once the film dissolves, the intestinal fluid surrounding the capsule enters the electrode cavity. Due to the electrolytic composition of the intestinal fluid, a small current will be able to pass from the output electrode, through the intestinal fluid, to the input electrode, causing it to read as a high value. Code from the microcontroller detects this input change and activates the RF transmission, which is initially turned off to conserve battery power.

A simulated intestinal fluid mimicking the osmolarity of real intestinal fluid was used to test this feature of the device. A 120 millimolar (mM) saltwater solution was created (in imitation of human small bowel (SB) fluid. With the RF transmission initially disabled, the electrodes were exposed to the simulated SB fluid to see if RF transmission would be activated. A Eudragit® L 100-55 polymer film 36 (which dissolves in solution with pH above 7.0) was then placed in the assembled location shown in FIG. 2, and the enclosed capsule was exposed sequentially to a gastric simulated environment of pH 2.5 and then the simulated SB fluid. The hypotheses were that the film would protect the electrodes in the stomach conditions, keeping the device in its idle state, but that once placed in the simulated small intestinal fluid, the film would dissolve, exposing and connecting the electrodes through the intestinal fluid, activating RF data transmission.

When the electrodes were exposed to the 120 mM solution mimicking the osmolarity of the small bowel, the microcontroller's interrupt was successfully triggered, resulting in the activation of RF data transmission to the receiving unit. Follow-up testing of the polymer film's integration capability also proved successful as the electrodes were kept guarded and dry in the acidic simulated gastric environment, yet were exposed once the film dissolved in the simulated SB environment. This testing confirms the integration capability of the odometer technology with new and pre-existing medical capsule technologies.

### Example 5

Benchtop testing was conducted to observe the behavior and localization ability of a capsule with multiple subsystems integrated together. To simulate an intestinal environment, 4 m of 1.5 inch (38.1 mm) diameter plastic tubing (U-LINE, PN: S-15162) was marked at 10 cm increments and filled with water. The fully assembled capsule was placed inside the tubing opening with the end of the suture thread adhered to a 3D printed anchoring fixture acting as the anchor inside the stomach. The capsule was moved and palpated by hand along the length of the tubing. To simulate an actual intestinal environment, the capsule was intermittently moved slightly in a retrograde direction, as well as through curves and up to 90-degree bends in the tubing. The transmitted count of spool revolutions at each 10 cm marking was recorded and inputted into the distance conversion model, which was then used to evaluate the average localization error. This was repeated for a total of three trials.

The localization results from testing with the complete assembled capsule inside plastic tubing is shown in Table 4. These results have slightly higher error values than seen in the initial benchtop testing. Potential sources of extra error include the markings on the plastic tubing not being accurately measured, or the capsule's location not being exactly at the location of a marking when data is recorded for that distance.

TABLE 4
Benchtop Testing Results

| Trial | Average Localization | Standard |
|---|---|---|
| Number | Error (cm) | Deviation (cm) |
| 1 | 2.79 | 1.49 |
| 2 | 3.87 | 2.29 |
| 3 | 1.89 | 1.27 |

The examples above serve to illustrate the capability of the systems and methods described herein when compared to existing localization technologies. Although the systems and methods discussed herein can be integrated with existing localization technologies to form hybrid systems and methods that have advantages over existing systems and methods, it is worth illustrating some of the advantages of the present systems and methods over such existing systems and methods (when used in accordance with traditional technologies). Thus, Table 5 provides an overview of the comparison between the systems and methods implementing odometry as described herein and other potential systems and methods for capsule localization. Indeed, the results show that the odometry systems and methods function as well or better than other methods in their ability to accurately and independently localize the capsule in a variety of environments.

TABLE 5
Localization Technology Comparison

| | | Distance | Independent | |
|---|---|---|---|---|
| | Provides | Tracking | of | External |
| Localization | Linear | Error | External | Mechanical |
| Method | Distance | (over 1 m) | Input | Parts |
| Odometry | Y | 0.7% | Y | N |
| OdoCapsule | Y | 6%* | Y | Y |
| Electromagnetic | N | N/A | N | N |
| Magnetism | N | N/a | N | N |
| Optical | Y | Not Tested | N | N |

*This device's error is accumulative, so error would increase over longer intestinal distances

Any and all of the components in the figures, embodiments, implementations, instances, cases, methods, applications, iterations, and other parts of this disclosure can be combined, mixed, substituted, or otherwise used together (in whole or in part) in any suitable manner. Additionally, any component can be removed, separated from other components, modified with or without modification of like components, or otherwise altered together or separately from anything else disclosed herein.

As used herein, the singular forms “a”, “an”, “the”, and other singular references include plural referents, and plural references include the singular, unless the context clearly dictates otherwise. For example, reference to a window includes reference to one or more windows, and reference to magnets includes reference to one or more magnets. In addition, where reference is made to a list of elements (e.g., elements a, b, and c), such reference is intended to include any one of the listed elements by itself, any combination of less than all of the listed elements, and/or a combination of all of the listed elements. Moreover, the term “or” by itself is not exclusive (and therefore may be interpreted to mean “and/or”) unless the context clearly dictates otherwise. Similarly, the term “and” by itself is not exclusive (and therefore may be interpreted to mean “and/or”) unless the context clearly dictates otherwise. Furthermore, the terms “including”, “i.e.,”, “having”, “such as”, “for example”, “e.g.”, and any similar terms are not intended to limit the disclosure, and may be interpreted as being followed by the words “without limitation”.

In addition, as the terms “on”, “disposed on”, “attached to”, “connected to”, “coupled to”, etc. are used herein, one object (e.g., a material, element, structure, member, etc.) can be on, disposed on, attached to, connected to, or otherwise coupled to another object—regardless of whether the one object is directly on, attached, connected, or coupled to the other object, or whether there are one or more intervening objects between the one object and the other object. Also, directions (e.g., “front”, “back”, “on top of”, “below”, “above”, “top”, “bottom”, “side”, “up”, “down”, “under”, “over”, “upper”, “lower”, “lateral”, “right-side”, “left-side”, “base”, etc.), if provided, are relative and provided solely by way of example and for ease of illustration and discussion and not by way of limitation.

The described systems and methods may be embodied in other specific forms without departing from their spirit or essential characteristics. The described embodiments, examples, and illustrations are to be considered in all respects only as illustrative and not restrictive. The scope of the described systems and methods is, therefore, indicated by the appended claims rather than by the foregoing description. All changes that come within the meaning and range of equivalency of the claims are to be embraced within their scope. Moreover, any component and characteristic from any embodiments, examples, and illustrations set forth herein can be combined in any suitable manner with any other components or characteristics from one or more other embodiments, examples, and illustrations described herein.

What is claimed is:

1. An ingestible medical capsule configured to measure a distance traveled by the ingestible medical capsule through a gastrointestinal tract of a subject, the ingestible medical capsule comprising:
an anchor configured to associate with a fixed anatomical feature of the subject; and
a capsule body coupled to the anchor via a strand, the capsule body being configured to travel a distance away from the anchor along the gastrointestinal tract of the subject and to measure the distance using the strand as a reference.

2. The ingestible medical capsule of claim 1, wherein the ingestible medical capsule is configured to perform an action at a certain point along the gastrointestinal tract, wherein the action includes at least one of: administering a medication; collecting a sample; capturing information; and transmitting data.

3. The ingestible medical capsule of claim 2, wherein the ingestible medical capsule is configured to perform the action in response to the capsule body traveling a certain distance away from the anchor.

4. The ingestible medical capsule of claim 1, wherein the anchor is initially associated with the capsule body, and wherein the anchor is configured to dissociate from the capsule body after the subject ingests the ingestible medical capsule.

5. The ingestible medical capsule of claim 4, further comprising an enteric coating configured to dissolve after the subject ingests the ingestible medical capsule to trigger a dissociation of the anchor from the capsule body.

6. The ingestible medical capsule of claim 1, wherein the fixed anatomical feature of the subject is selected from at least one of: (i) a face, (ii) cheek, (iii) chin, (iv) tooth, (v) tongue, and (vi) mouth of the subject.

7. The ingestible medical capsule of claim 4, wherein the anchor is configured to expand after dissociating from the capsule body.

8. The ingestible medical capsule of claim 1, wherein the anchor is configured to anchor on a pyloric valve of the subject.

9. The ingestible medical capsule of claim 1, wherein the anchor is configured to stop anchoring and to pass through the gastrointestinal tract of the subject after an interval of time has passed, wherein the interval of time is at least 24 hours.

10. The ingestible medical capsule of claim 1, further comprising a spool housed within the capsule body, wherein the strand is initially wound around the spool, and wherein the capsule body is configured to track an unwinding of the strand from the spool.

11. An ingestible medical capsule comprising:
a spool;
a strand configured to unspool from the spool as the ingestible medical capsule travels through a gastrointestinal tract of a subject;
a detector configured to measure a length of the strand that unspools from the spool; and
a transmitter configured to communicate information about the length of the strand that unspools from the spool to a user external to the subject.

12. The ingestible medical capsule of claim 11, wherein the spool is configured to revolve as the strand unspools from the spool.

13. The ingestible medical capsule of claim 12, wherein the detector comprises:
a magnet coupled to the spool; and
a Hall sensor configured to sense a change in a magnetic field produced by the magnet as the spool revolves.

14. The ingestible medical capsule of claim 13, further comprising a controller electronically coupled to the Hall sensor, the controller being configured to count revolutions of the spool based on data received from the Hall sensor.

15. The ingestible medical capsule of claim 14, further comprising a printed circuit board configured to electronically couple to the Hall sensor, the controller, the transmitter, and a power source.

16. A method of localizing an ingestible medical capsule within a gastrointestinal tract of a subject, the method comprising:
coupling a capsule body of the ingestible medical capsule to an anchor via a strand; and
measuring a linear distance between the capsule body and the anchor to determine a distance traveled by the capsule body.

17. The method of claim 16, further comprising associating the anchor with the capsule body and coating the anchor and the capsule body with an enteric coating, wherein the enteric coating is configured to dissolve in the gastrointestinal tract of the subject, thereby causing the anchor to dissociate from the capsule body.

18. The method of claim 17, wherein the anchor is selectively configured not to pass through a pyloric valve of the subject.

19. The method of claim 16, further comprising spooling the strand around a spool and disposing the spool within a housing of the capsule body.

20. The method of claim 19, wherein the measuring the linear distance between the capsule body and the anchor to determine the distance traveled by the capsule body comprises detecting a number of revolutions of the spool as the strand unspools from the spool.
